(19) United States
(12) Patent Application Publication
(10) Pub. No.: US 2019/0117196 A1
(43) Pub. Date: Apr. 25, 2019

(54) ULTRASOUND DIAGNOSTIC APPARATUS AND ULTRASOUND PROBE

(71) Applicant: Canon Medical Systems Corporation, Otawara-shi (JP)

(72) Inventor: Jaeho CHOI, Utsunomiya (JP)

(73) Assignee: Canon Medical Systems Corporation, Otawara-shi (JP)

(21) Appl. No.: 16/163,704

(22) Filed: Oct. 18, 2018

(30) Foreign Application Priority Data
Oct. 19, 2017 (JP) 2017-202727
Oct. 16, 2018 (JP) 2018-195190

(51) Int. Cl. A61B 8/08; A61B 8/00

(52) U.S. Cl. CPC A61B 8/5207; A61B 8/5246; A61B 8/14; A61B 8/461; A61B 8/4444; A61B 8/4254

(57) ABSTRACT

According to one embodiment, an ultrasound diagnostic apparatus includes a transmission/reception circuitry, a switching power supply, and control circuitry. The transmission/reception circuitry transmits ultrasound to a subject in a predetermined repetition cycle and receives an echo signal from the subject. The switching power supply generates a voltage by switching in accordance with a switching frequency, and supplies the transmission/reception circuitry with the voltage. The control circuitry changes the switching frequency by a predetermined change width in the predetermined repetition cycle.

### CROSS-REFERENCE TO RELATED APPLICATIONS

This application is based upon and claims the benefit of priority from the prior Japanese Patent Application No. 2017-202727, filed Oct. 19, 2017 and No. 2018-195190, filed Oct. 16, 2018, the entire contents of both which are incorporated herein by reference.

### FIELD

Embodiments described herein relate generally to an ultrasound diagnostic apparatus and an ultrasound probe.

### BACKGROUND

As a power supply for ultrasound diagnostic apparatuses of recent years, a switching power supply which has high conversion efficiency and is low-cost is sometimes used. The switching power supply is a power supply that generates given different voltages by switching on and off a transistor. The number of times the transistor is switched in one second is called a switching frequency.

In an ultrasound diagnostic apparatus using the switching power supply, the switching frequency may coincide with an integral multiple of a pulse repetition frequency at which ultrasound pulses are transmitted when a scan is performed. In such a case, switching noises attributed to switching may be shown on an ultrasound image based on ultrasound image data generated by, for example, performing a brightness- (B-) mode scan or a motion- (M-) mode scan. In particular, when a plurality of echo signals obtained by transmitting ultrasound multiple times in the same direction (to the same scan line) are summed, and ultrasound image data is generated based on the sum of the echo signals as in a pulse inversion method or a combination focus method, switching noises shown on the ultrasound image based on the generated ultrasound image data become prominent.

### BRIEF DESCRIPTION OF THE DRAWINGS

FIG. 1 is a diagram showing a configuration of an ultrasound diagnostic apparatus according to a first embodiment.

FIG. 2 is a diagram for illustrating the method for controlling the switching frequency according to the first embodiment.

FIG. 3 is a timing chart showing the relationship between the transmission timing of ultrasound pulses and the supply timing of switching clocks according to the first embodiment.

FIG. 4 is a diagram showing the relationship between the switching clocks generated in respective cycles based on the PRI with reference to the transmission timing of ultrasound pulses defined by the PRI.

FIG. 5 is a diagram for illustrating the duty ratio of each switching clock of the case where the control circuitry according to the first embodiment changes the switching frequency.

FIG. 6 is a diagram showing a B-mode image displayed on a display by the ultrasound diagnostic apparatus 1 according to the first embodiment.

FIG. 7 is a diagram showing a B-mode image displayed on the display when the switching frequency is not controlled.

FIG. 8 is a diagram showing a configuration of an ultrasound diagnostic apparatus according to a second embodiment.

### DETAILED DESCRIPTION

In general, according to one embodiment, an ultrasound diagnostic apparatus includes a transmission/reception circuitry, a switching power supply, and control circuitry. The transmission/reception circuitry transmits ultrasound to a subject in a predetermined repetition cycle and receives an echo signal from the subject. The switching power supply generates a voltage by switching in accordance with a switching frequency, and supplies the transmission/reception circuitry with the voltage. The control circuitry changes the switching frequency by a predetermined change width in the predetermined repetition cycle.

Hereinafter, embodiments will be described with reference to drawings.

### First Embodiment

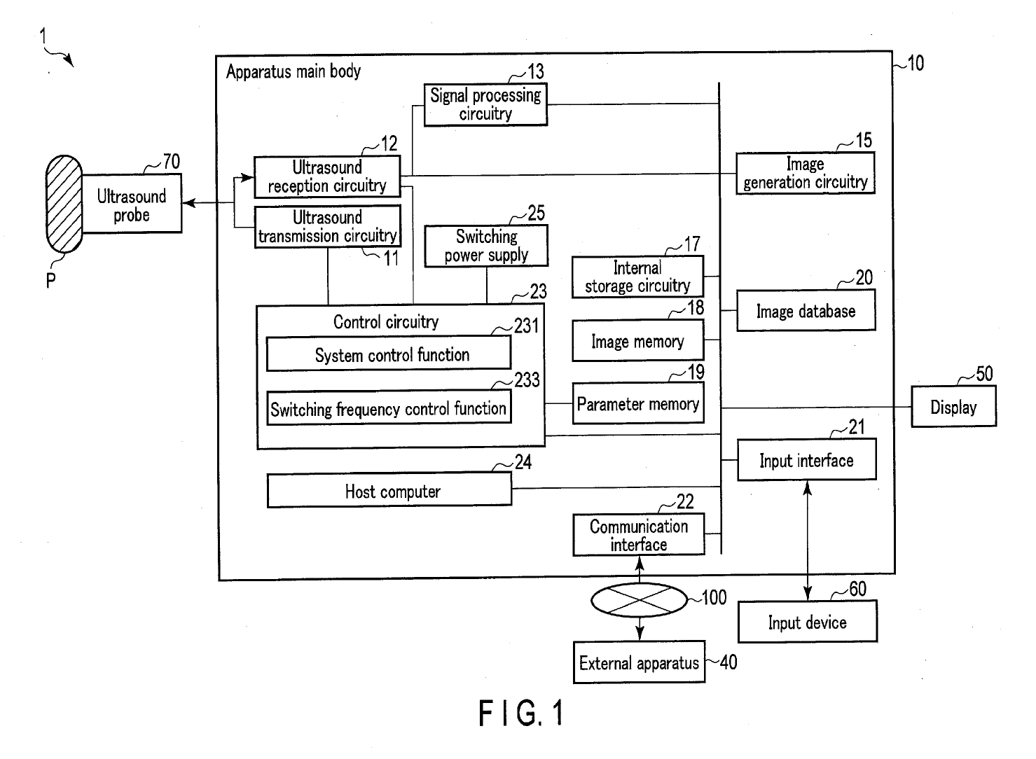

FIG. 1 is a diagram showing an example of the functional configuration of an ultrasound diagnostic apparatus 1 according to a first embodiment. As shown in FIG. 1, the ultrasound diagnostic apparatus 1 includes an apparatus main body 10, an ultrasound probe 70, a display 50, and an input device 60. The apparatus main body 10 is connected to an external apparatus 40 via a network 100. The apparatus main body 10 is also connected to the display 50 and the input device 60.

The ultrasound probe 70 is, for example, a one-dimensional array probe in which a plurality of piezoelectric vibrators are arranged in a predetermined direction, a two-dimensional array probe in which a plurality of piezoelectric vibrators are arranged in a two-dimensional matrix, or a mechanical four-dimensional probe capable of performing an ultrasound scan while mechanically sweeping a piezoelectric vibrator line in directions orthogonal to the alignment direction.

The ultrasound probe 70 includes, for example, a plurality of piezoelectric vibrators, a matching layer provided in each piezoelectric vibrator, and a backing material for preventing backward propagation of ultrasound from the piezoelectric vibrators. The ultrasound probe 70 is detachably attached to the apparatus main body 10. The piezoelectric vibrators generate ultrasound based on a drive signal supplied from ultrasound transmission circuitry 11 included in the apparatus main body 10. The ultrasound probe 70 may be provided with a button pressed when, for example, performing offset processing or freezing an ultrasound image.

When ultrasound is transmitted from the ultrasound probe 70 to a subject P, the transmitted ultrasound is reflected by one after another of discontinuous surfaces having acoustic impedances in body tissue of the subject P, and is received at the piezoelectric vibrators included in the ultrasound probe 70 as a reflected wave (echo). The ultrasound probe 70 converts the received reflected wave into an electrical signal (reflected wave signal). The reflected wave signal may be reworded as an echo signal. The amplitude of the reflected wave signal depends on the difference in acoustic impedances of the discontinuous surfaces by which ultrasound is reflected. The reflected wave signal of the case where a transmitted ultrasound pulse is reflected by a moving blood flow or a moving surface of a cardiac wall or the like is subjected to a frequency shift by the Doppler effect while depending on the velocity component in the ultrasound transmission direction of the moving object.

The apparatus main body 10 shown in FIG. 1 is an apparatus that generates an ultrasound image based on the reflected wave signal output from the ultrasound probe 70. The apparatus main body 10 includes ultrasound transmission circuitry 11, ultrasound reception circuitry 12, signal processing circuitry 13, image generation circuitry 15, internal storage circuitry 17, an image memory 18, a parameter memory 19, an image database 20, an input interface 21, a communication interface 22, control circuitry 23, a host computer 24, and a switching power supply 25, as shown in FIG. 1.

The ultrasound transmission circuitry 11 is a processor that supplies the ultrasound probe 70 with a drive signal. The ultrasound transmission circuitry 11 is realized by, for example, a trigger generation circuit, a delay circuit, and a pulser circuit. The trigger generation circuit repeatedly generates a rate pulse for forming transmission ultrasound at a predetermined rate frequency, i.e., a pulse repetition frequency (PRF), under the control of the control circuitry 23. The delay circuit provides each rate pulse generated by the trigger generation circuit with a delay time for each piezoelectric vibrator necessary for converging ultrasound generated by the ultrasound probe 70 in a beam form and determining transmission directivity. The pulser circuit applies a drive signal (drive pulse) to the ultrasound probe 70 at times based on the rate pulse under the control of the control circuitry 23. By varying the delay time provided to each rate pulse by the delay circuit, the transmission direction from the piezoelectric vibrator surface can be freely adjusted.

The ultrasound reception circuitry 12 is a processor that performs various processes on the reflected wave signal output from the ultrasound probe 70 to generate a digitized reflected wave signal (hereinafter referred to as a received signal). The ultrasound reception circuitry 12 is realized by, for example, an amplifier circuit, an A/D (Analog to Digital) converter, a reception delay circuit, and an adder. The amplifier circuit performs a gain correction process by amplifying the reflected wave signal output from the ultrasound probe 70 for each channel. The A/D converter converts the gain-corrected reflected wave signal into a digital signal. The reception delay circuit provides the digital signal with a delay time necessary for determining reception directivity. The adder sums a plurality of digital signals each provided with a delay time. By the summation process of the adder, a received signal with an enhanced reflected component in a direction corresponding to the reception directivity is generated. The received signal includes amplitude information reflecting the acoustic impedance difference between tissues, and phase information reflecting movement of a body tissue, such as a motion or travel speed, etc.

The signal processing circuitry 13 is a processor that performs various types of signal processing on the received signal received from the ultrasound reception circuitry 12. The signal processing circuitry 13 performs an envelope wave detecting process, a log arithmic amplifying process, etc. on the received signal received from the ultrasound reception circuitry 12 to generate data that expresses signal intensity by brightness (B-mode data). The generated B-mode data is stored in a raw data memory (not shown) as B-mode raw data on a two-dimensional ultrasound scan line.

The signal processing circuitry 13 also performs a frequency analysis on the received signal received from the ultrasound reception circuitry 12 to extract a blood-flow signal and generate data obtained by extracting, from the blood-flow signal, information such as an average speed, dispersion, and power, on multiple points (Doppler data). The generated Doppler data is stored in a raw data memory (not shown) as Doppler raw data on a two-dimensional ultrasound scan line.

The image generation circuitry 15 is a processor capable of generating various ultrasound image data based on data generated by the signal processing circuitry 13. The image generation circuitry 15 generates B-mode image data based on the B-mode raw data stored in the raw data memory. A B-mode image based on the B-mode image data shows, for example, a form of a structure in the subject P. The B-mode image data has a pixel value (brightness value) reflecting, for example, characteristics of the ultrasound probe, such as sound convergence, and sound-field characteristics of an ultrasound beam (e.g., a transmitted and received beam). For example, B-mode image data has a relatively higher brightness in the vicinity of the focus of ultrasound in the scanned area than in the unfocused part.

The image generation circuitry 15 generates Doppler image data showing moving object information based on the Doppler raw data stored in the raw data memory. The Doppler image data is speed image data, dispersion image data, power image data, or image data of a combination of aforementioned data.

The image generation circuitry 15 converts (scan-converts) a scan line signal sequence of an ultrasound scan into, for example, a scan line signal sequence in a video format representatively used by television to generate ultrasound image data for display. Specifically, the image generation circuitry 15 performs a coordinate conversion corresponding to the form of the ultrasound scan by the ultrasound probe 70 to generate ultrasound image data for display.

The image generation circuitry 15 may perform various processes, such as dynamic range, brightness, contrast, y curve corrections, and an RGB conversion, on generated various ultrasound image data. The image generation circuitry 15 may add supplementary information, such as textual information of various parameters, a scale, or a body mark, to the generated various ultrasound image data.

The image generation circuitry 15 may generate a user interface (graphical user interface: GUI) for the operator (such as a person performing surgery) to input various instructions by the input interface 21, and display the GUI on the display 50. As the display 50, for example, a CRT display, a liquid crystal display, an organic EL display, an LED display, a plasma display, or any other display known in the relevant technical field may be used as appropriate. The display 50 may have a function of, for example, an informing section.

The internal storage circuitry 17 includes, for example, a magnetic or optical storage medium, or a processor-readable storage medium such as a semiconductor memory. The internal storage circuitry 17 stores, for example, a control program for executing ultrasound transmission and reception, a control program for performing image processing, a control program for performing display processing, and control programs for realizing various functions according to the present embodiment.

The internal storage circuitry 17 also stores diagnostic information (such as a patient's ID, and a doctor's observation), a diagnostic protocol, a body mark generation program, and a data group such as a conversion table in which the range of color data used for imaging is preset for each diagnostic site. The internal storage circuitry 17 may also store an anatomical picture, such as an atlas, concerning the structure of an organ in a living body.

The internal storage circuitry 17 stores various ultrasound image data generated at the image generation circuitry 15, in accordance with a storing operation input via the input interface 21. The internal storage circuitry 17 may store various ultrasound image data generated at the image generation circuitry 15 together with the operation order and operation time, in accordance with a storing operation input via the input interface 21. The internal storage circuitry 17 may transfer the stored data to an external apparatus via the communication interface 22.

The image memory 18 includes, for example, a magnetic or optical storage medium, or a processor-readable storage medium such as a semiconductor memory. The image memory 18 stores image data for display generated by the image generation circuitry 15. The image data stored here is, for example, image data representing an image actually displayed on the display 50. The image memory 18 stores image data corresponding to a plurality of frames immediately before a freeze operation input via the input interface 21. The image data stored in the image memory 18 is, for example, continuously displayed (cine-displayed). The image displayed on the display 50 may include, for example, an image based on ultrasound image obtained by an ultrasound scan, and an image based on diagnostic image data obtained by another modality, such as computed tomography (CT) image data, magnetic resonance (MR) image data, X-ray image data, or position emission tomography (PET) image data.

The image memory 18 can also store data generated by the signal processing circuitry 13. The B-mode data and Doppler data stored in the image memory 18 can be taken out by the operator, for example, after diagnosis, and can be turned into ultrasound image data for display through the image generation circuitry 15.

The parameter memory 19 includes, for example, a storage medium readable at high speed by a processor, such as a semiconductor memory. The parameter memory 19 is, for example, a main memory. The parameter memory 19 stores a parameter (hereinafter referred to as a control parameter) necessary for performing an ultrasound scan. The control parameter includes, for example, frame information, vector information, beam information, a transmitter element position, a transmission delay, a transmit aperture, header information, a digital filter coefficient, probe selection data, and gain data.

The image database 20 stores image data transferred from the external apparatus 40. For example, the image database 20 obtains and stores historical image data from the external apparatus 40 concerning the same patient obtained from the past medical examination. The historic image data includes ultrasound image data, CT image data, MR image data, PET-CT image data, PET-MR image data, and X-ray image data. The historic image data is stored as, for example, volume data and rendering image data.

The image database 20 may store desired image data by reading image data stored in a storage medium such as an MO, a CD-R, or a DVD.

The input interface 21 receives various instructions from the operator via the input device 60. The input device 60 includes, for example, a mouse, a keyboard, a panel switch, a slider switch, a dial switch, a trackball, a rotary encoder, an operation panel, and a touch command screen (TCS). The input device 60 includes a switch group for switching various imaging modes including an ultrasound transmission/reception scheme, and received signal processing scheme, etc. The switch group may be not only a mechanical device, such as a dial switch or a track ball, but also an operation panel image displayed on a TCS, or an operation panel image displayed on a second console in the external apparatus 40.

The input interface 21 is connected to the host computer 24 via, for example, a bus, converts an operation instruction input by the operator into an electrical signal, and outputs the electrical signal to the host computer 24. In this specification, the input interface 21 is not limited to the one for connection to a physical operational component, such as a mouse or a keyboard. For example, processing circuitry that receives, as a wireless signal, an electrical signal corresponding to an operation instruction input from an external input device provided separately from the ultrasound diagnostic apparatus 1, and outputs the electrical signal to the host computer 24, is also an example of the input interface 21.

The communication interface 22 is connected to the external apparatus 40 via, for example, the network 100, and performs data communication with the external apparatus 40. The external apparatus 40 is, for example, a database of a picture archiving and communication system (PACS) which is a system that manages data of various medical images, and a database of an electronic health record system which manages electronic health records accompanied with medical images. The external apparatus 40 is also, for example, various medical image diagnostic apparatuses other than the ultrasound diagnostic apparatus 1 according to the present embodiment, such as an X-ray CT apparatus, a magnetic resonance imaging (MRI) apparatus, a nuclear medicine diagnostic apparatus, and an X-ray diagnostic apparatus. The standard of communication with the external apparatus 40 may be any standard, but is, for example, digital imaging and communication medicine (DICOM).

The control circuitry 23 is, for example, a processor that controls operations relating to an ultrasound scan. The control circuitry 23 performs an operation program stored in the internal storage circuitry 17 to realize a function corresponding to the operation program. Specifically, the control circuitry 23 has a system control function 231, and a switching frequency control function 233.

The system control function 231 and the switching frequency control function 233 are not necessarily incorporated in the internal storage circuitry 17 as control programs. The system control function 231 and the switching frequency control function 233 may be incorporated in, for example, the control circuitry 23. The system control function 231 and the switching frequency control function 233 may also be incorporated in, for example, the apparatus main body 10 as dedicated hardware circuits capable of executing the respective functions.

The system control function 231 is a function of performing various operations based on various instructions from the host computer 24. When the system control function 231 is performed, the control circuitry 23 receives a start instruction to start an ultrasound scan in each imaging mode from the host computer 24. At this time, the control circuitry 23 also receives a beam number, a frame rate, a depth, etc. as input information. The control circuitry 23 generates an ultrasound pulse at a predetermined PRF based on the received start instruction, beam number, frame rate, depth, etc.

Based on the received input information, the control circuitry 23 sets control parameters for the ultrasound transmission circuitry 11 and the ultrasound reception circuitry 12. Specifically, the control circuitry 23, for example, reads transmission position information, a transmit aperture, a transmission delay, etc. from the parameter memory 19, and sets the read transmission position information, transmit aperture, transmission delay, etc. in the ultrasound transmission circuitry 11 together with the value of the PRF. The control circuitry 23 also reads a receive aperture, a reception delay, etc. from the parameter memory 19, and sets the read receive aperture, reception delay, etc. in the ultrasound reception circuitry 12.

The control circuitry 23 controls the ultrasound transmission circuitry 11 and the ultrasound reception circuitry 12 based on the set control parameters, and performs an ultrasound scan corresponding to each imaging mode. Specifically when receiving from the host computer 24 a start instruction to start a B-mode ultrasound scan, for example, the control circuitry 23 controls the ultrasound transmission circuitry 11 and the ultrasound reception circuitry 12 to perform the B-mode scan. For example, when receiving from the host computer 24 a start instruction to start an M-mode ultrasound scan, the control circuitry 23 controls the ultrasound transmission circuitry 11 and the ultrasound reception circuitry 12 to perform the M-mode scan.

When the control circuitry 23, for example, receives from the host computer 24 a performance instruction to perform a pulse inversion (PI) in a state where the B mode is selected, the control circuitry 23 controls the ultrasound transmission circuitry 11 and the ultrasound reception circuitry 12 to repeatedly perform a B-mode scan as described below. Namely, the control circuitry 23 controls the ultrasound transmission circuitry 11, and consecutively transmits two ultrasound waves having a phase difference of 180 degrees in the same direction from the ultrasound probe 70 to the subject P. Then, the control circuitry 23 controls the ultrasound reception circuitry 12, receives two reflected wave signals generated by the two ultrasound transmissions, performs various processes on the received two reflected wave signals, and generates two received signals having a phase difference of 180 degrees.

For example, when the control circuitry 23 receives from the host computer 24 a performance instruction to perform a combination focus in the state where the B mode is selected, the control circuitry 23 controls the ultrasound transmission circuitry 11 and the ultrasound reception circuitry 12 to repeatedly perform the B-mode scan as described below. Namely, the control circuitry 23 controls the ultrasound transmission circuitry 11, and transmits a plurality of ultrasound waves in the same direction from the ultrasound probe 70 to respective transmission focuses of the subject P. Then, the control circuitry 23 controls the ultrasound reception circuitry 12, receives a plurality of reflected wave signals generated in response to the ultrasound transmissions, performs various processes on the received reflected wave signals, and generates a plurality of received signals of different transmission focuses.

The switching frequency control function 233 is a function of controlling the switching frequency for determining the timing of the switching operation of the switching power supply 25 to be described later. This function can be reworded as a function of generating a switching clock at a predetermined frequency, and supplying the generated switching clock to the switching power supply 25. When the switching frequency control function 233 is performed, the control circuitry 23 controls the switching frequency so that the phase differences between the PRF and switching frequencies are scattered. For example, the control circuitry 23 gradually changes the switching frequency in a cycle based on a pulse repetition interval (PRI) corresponding to the PRF. Specifically, the control circuitry 23 changes the switching frequency in the repetition cycle by a preset change width, such as 1% of the switching frequency before a change. The change width is, for example, a width that inhibits the output voltage supplied by the switching power supply 25 from being unstable. The control circuitry 23 can receive a given value, for example, via the input interface 21, and set the received value as the change width. The change width of the switching frequency is not limited to 1%, and may be, for example, 0.5% or 2%.

The host computer 24 includes a processor, and functions as the nerve center of the ultrasound diagnostic apparatus 1. The host computer 24 receives various instructions from the operator or the like via the input interface 21. The host computer 24 inputs the received various instructions to the control circuitry 23. The host computer 24 controls the signal processing circuitry 13 and the image generation circuitry 15 in accordance with the received instruction, and generates predetermined ultrasound image data based on the received signals generated at the ultrasound reception circuitry 12.

For example, when the host computer 24 receives, via the input interface 21, a performance instruction to perform a pulse inversion in the state where the B mode is selected, the host computer 24 inputs the received performance instruction to the control circuitry 23. The host computer 24 controls the signal processing circuitry 13 and the image generation circuitry 15, and sums, for example, two received signals generated by the ultrasound reception circuitry 12, i.e., two received signals having a phase difference of 180 degrees. Accordingly, the fundamental wave component is suppressed, and a harmonic signal mainly corresponding to the second harmonic component is generated. Then, the host computer 24 generates ultrasound image data based on the generated harmonic signal.

When the host computer 24 receives, via the input interface 21, a performance instruction to perform a combination focus in the state where the B mode is selected, the host computer 24 inputs the received performance instruction to the control circuitry 23. The host computer 24 controls the signal processing circuitry 13 and the image generation circuitry 15, and sums, for example, a plurality of received signals generated by the ultrasound reception circuitry 12, i.e., a plurality of received signals of different transmission focuses. Accordingly, a received signal is generated, by which from superficial regions to deep regions are focused. Then, the host computer 24 generates ultrasound image data based on the generated received signals.

The host computer 24 displays an ultrasound image based on the generated ultrasound image data on the display 50.

The switching power supply 25 includes, for example, an AC/DC converter circuit, or a DC/DC converter circuit. The switching power supply 25 turns on and off the transistor in accordance with the switching clocks supplied from the control circuitry 23 to generate a DC voltage having a predetermined voltage value from an AC voltage or DC voltage input from a commercial power supply (not shown). The switching power supply 25 supplies the generated DC voltage to each circuitry of the ultrasound diagnostic apparatus 1, for example, the ultrasound transmission circuitry 11 and the ultrasound reception circuitry 12.

Next, an example of the method for controlling the switching frequency by the switching frequency control function 233 of the control circuitry 23 according to the present embodiment will be described with reference to drawings.

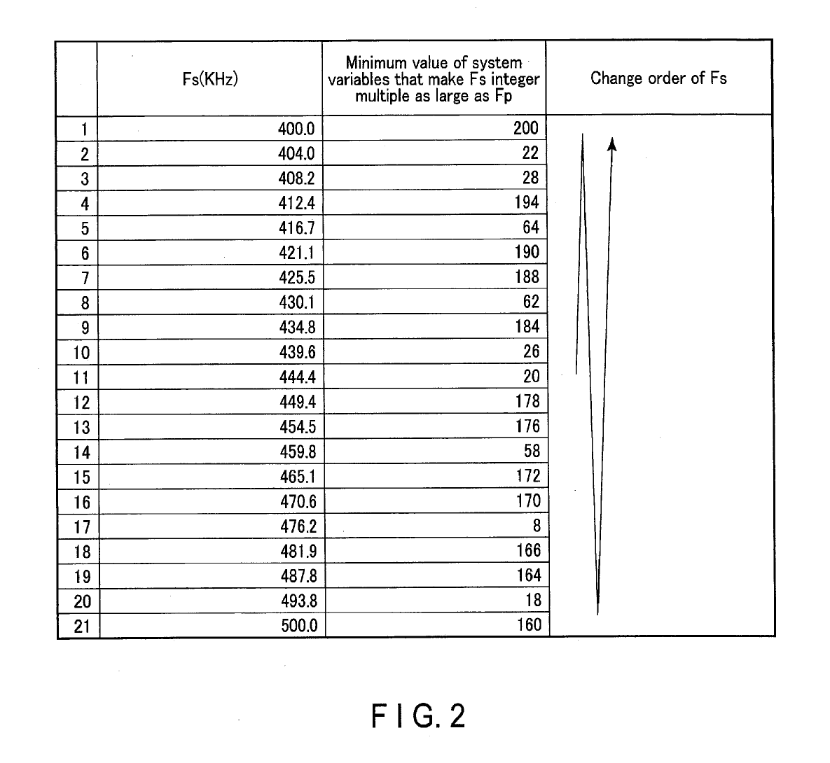

FIG. 2 is a diagram for illustrating an example of the method for controlling the switching frequency according to the present embodiment. In the following description, let us assume that a performance instruction to perform a combination focus in the state where the B mode is selected, and that a B-mode scan is performed on each of four transmission focuses. In addition, the following description will be provided while taking the case where the permissible frequency range of the switching power supply 25 is from 400 KHz to 500 KHz as an example. The number of transmission focuses may be any integer equal to or larger than 2.

The method for controlling the switching frequency according to the present embodiment is applicable to scans other than the B-mode scan using the combination focus method. For example, the method for controlling the switching frequency according to the present embodiment is applicable to a method of summing a plurality of echo signals obtained by transmitting ultrasound in the same direction (to the same scan line) multiple times, and generating ultrasound image data based on the sum of the echo signals. Namely, the method for controlling the switching frequency according to the present embodiment is applicable to, for example, an M-mode scan using the combination focus method, a B-mode scan using the pulse inversion method, and an M-mode scan using the pulse inversion method.

The PRI defining the transmission timing of ultrasound pulses is set to N (N is a positive integer) times as large as the cycle based on the operation frequency (hereinafter referred to as a system clock frequency) of, for example, the ultrasound transmission circuitry 11 or the ultrasound reception circuitry 12. N is called a system variable, and varies in accordance with the depth of transmission or reception of an ultrasound pulse. In the following description, let us assume that the system clock frequency is, for example, 1.27 MHz. When the system clock frequency is 1.27 MHz, and N at the time of performing the B-mode scan is 20, the PRF Fp is 1.27 MHz/20=63.5 KHz.

FIG. 2 shows the switching frequency Fs (KHz), the minimum value of the system variables which make Fs an integer multiple as large as the PRF Fp (KHz), and the changing order of Fs. The minimum value of the system variables which make Fs an integer multiple as large as Fp is the minimum value of the system variables which may cause a switching noise to be superimposed on the ultrasound image. In the case of FIG. 2, the initial value of the switching frequency is set to 444.4 KHz. The initial value of the switching frequency is preset, for example, via the input interface 21. The switching frequency 444.4 KHz is approximately seven times, which is an integer multiple, as large as the PRF 63.5 KHz; therefore, a switching noise attributed to switching is shown on the ultrasound image obtained by the B-mode scan or M-mode scan.

The control circuitry 23 according to the present embodiment determines the maximum value and minimum value of the switching frequency so that they fall within the range, for example, from approximately minus 10% to plus 10% of the initial value. Accordingly, as shown in FIG. 2, the maximum value of the switching frequency is determined to be, for example, 500 KHz with reference to the initial value 444.4 KHz. On the other hand, the minimum value of the switching frequency is determined to be, for example, 400 KHz.

If a combination focus using four transmission focuses is performed by a common ultrasound diagnostic apparatus by using the above setting, reflected wave signals are respectively detected from four different depths in the same direction. Since the switching frequency 444.4 KHz is approximately seven times, which is an integer multiple, as large as the PRF 63.5 KHz, the detected four reflected wave signals include switching noises generated in the same time phase. Therefore, if the received signals based on the four reflected wave signals are summed, the switching noises included in the four reflected wave signals are also summed in the same time phase, and the switching noise shown on the generated B-mode image becomes prominent.

Therefore, the control circuitry 23 according to the present embodiment changes the switching frequency by a predetermined change width, for example, in a cycle based on the PRI. At this time, the switching frequency is changed, for example, based on the transmission timing of ultrasound pulses. The change timing of the switching frequency is not necessarily based on the transmission timing of ultrasound pulses, and may be any timing as long as it follows the cycle based on the PRI.

In the example shown in FIG. 2, the control circuitry 23 gradually changes the switching frequency in the cycle based on the PRI that is set by setting the system variable to 20. For example, the switching frequency is changed in stages as follows:

444.4 KHz→439.6 KHz→434.8 KHz→430.1 KHz→425.5 KHz→421.1 KHz→416.7 KHz→412.4 KHz→408.2 KHz→404.0 KHz→400.0 KHz→404.0 KHz→408.2 KHz→412.4 KHz→416.7 KHz→421.1 KHz→425.5 KHz→430.1 KHz→434.8 KHz→439.6K Hz→444.4 KHz→449.4 KHz→454.5 KHz→459.8 KHz→465.1 KHz→470.6 KHz→476.2 KHz→481.9 KHz→4487.8 KHz→4493.8 KHz→500.0 KHz→493.8 KHz→487.8 KHz→481.9 KHz→476.2 KHz→470.6 KHz→465.1 KHz→459.8 KHz→4454.5 KHz→449.4 KHz. The control circuitry 23 repeatedly performs such a change control.

According to the above control method, the switching frequency is 444.4 KHz in the first cycle based on the PRI; accordingly, the switching frequency is an integer multiple as large as the PRF 63.5 KHz. In the second and subsequent cycles, the switching frequency is not an integer multiple as large as the PRF other than the cycle in which the switching frequency is 444.4 KHz. Accordingly, cycles in which the switching frequency is an integer multiple as large as the PRF never become consecutive. Namely, it becomes possible to prevent switching noises included in reflected wave signals from being summed in the same phase in, for example, the pulse inversion method or the combination focus method.

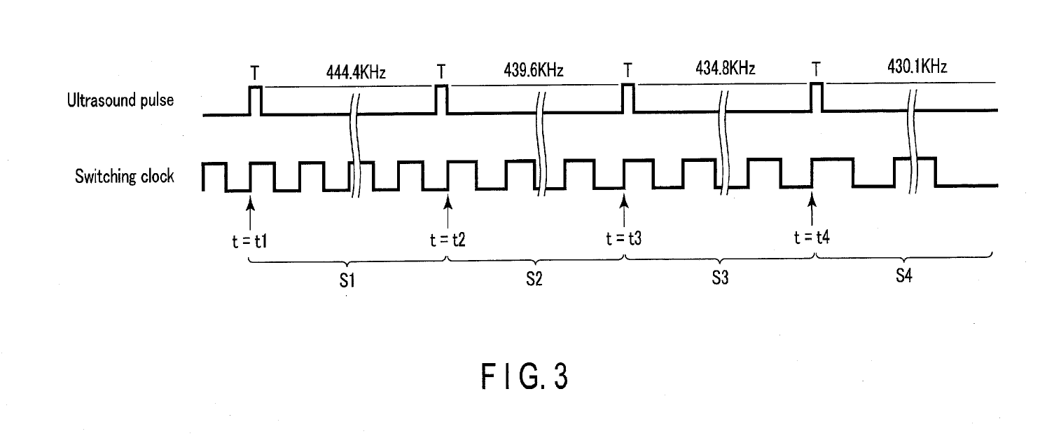

Next, the relationship between the transmission timing of ultrasound pulses and the supply timing of switching clocks at the time when the control circuitry 23 of the ultrasound diagnostic apparatus 1 according to the present embodiment changes the switching frequency will be described with reference to FIG. 3. FIG. 3 is a timing chart showing an example of the relationship between the transmission timing of ultrasound pulses and the supply timing of switching clocks according to the present embodiment. The upper waveform shown in FIG. 3 shows an example of the transmission waveform of four ultrasound pulses transmitted in accordance with the set PRI. T indicates the transmission timing of an ultrasound pulse defined by the PRI.

The lower waveform shown in FIG. 3 shows a transmission waveform of switching clocks t=t1, t=t2, t=t3, and t=t4 indicate times when the switching frequency is changed. t=t1, t=t2, t=t3, and t=t4 are each determined, for example, based on the transmission timing T of the corresponding ultrasound pulse. The time when the switching frequency is changed is not limited to the time based on the transmission timing T of an ultrasound pulse, and may be any time as long as it follows the cycle based on the PRI.

According to FIG. 3, the control circuitry 23 changes the switching frequency in the order of “444.4 KHz→439.6 KHz→434.8 KHz→430.1 KHz” by performing the switching frequency control function 233. Namely, the control circuitry 23 generates switching clock S1 for 444.4 KHz, switching clock S2 for 439.6 KHz, switching clock S3 for 434.8 KHz, switching clock S4 for 430.1 KHz and supplies them to the switching power supply 25 at respective times t=t1, t=t2, t=t3, and t=t4.

The system variable for determining the PRF that coincides with a switching frequency when multiplied by an integer varies between switching frequencies. Namely, the minimum value of the system variables which make Fs an integer multiple as large as Fp varies among switching frequencies. Specifically, the minimum value of the system variable for switching frequency 444.4 KHz is 20, that for switching frequency 439.6 KHz is 26, that for switching frequency 434.8 KHz is 184, and that for switching frequency 430.1 KHz is 62, and they all vary from one another.

In the above description referring to FIG. 2, the system variable is set to 20, and the PRF is set to 63.5 KHz. Therefore, when the switching frequency is changed by predetermined change widths based on the transmission timing T of ultrasound pulses, the minimum value of the system variables which may cause a switching noise varies among changed switching frequencies. Namely, the phases of four switching clocks corresponding to changed switching frequencies never match each other.

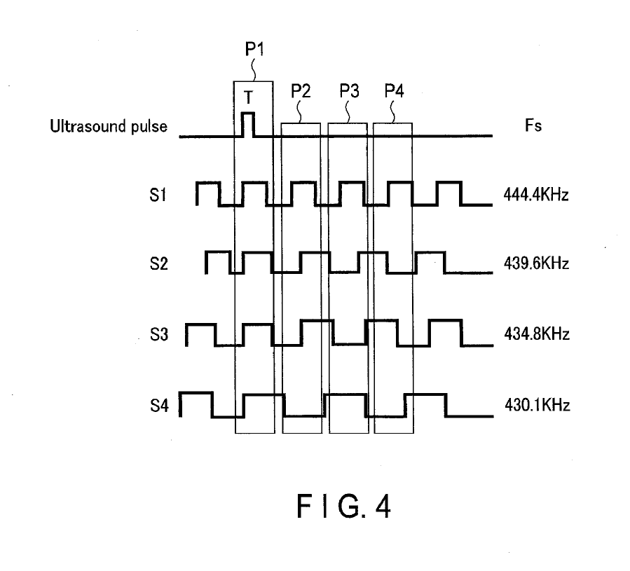

FIG. 4 is a diagram showing the relationship between switching clocks generated in respective cycles with reference to the time when an ultrasound pulse defined by the PRI rises. FIG. 4 shows the wave forms of switching clocks S1, S2, S3, and S4 by vertically aligning then with reference to the time when the ultrasound pulse rises.

In FIG. 4, in period P1 including the transmission timing T of an ultrasound pulse, the ultrasound transmission pulse, switching clock S1, switching clock S2, switching clock S3, and switching clock S4 are turned on at the same time. In contrast, the ultrasound transmission pulse is not turned off at the same time as each switching clock in period P1. In periods P2, P3, and P4, the ultrasound transmission pulse is not turned on or off at the same time as each switching clock. Accordingly, FIG. 4 shows that the phase differences between the ultrasound transmission pulse and respective switching clocks are scattered. Consequently, even when four received signals generated while respective switching clocks S1, S2, S3, and S4 are being supplied are summed, switching noises are not summed in the same phase. Therefore, an increase in the switching noise shown on the ultrasound image generated by summing a plurality of received signals can be inhibited.

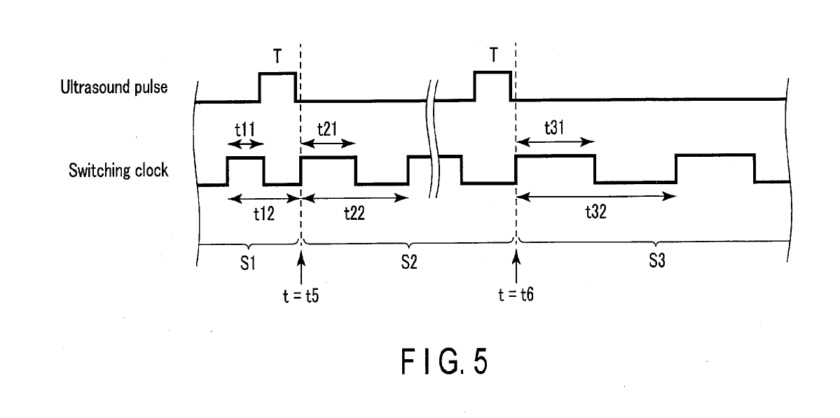

The ultrasound diagnostic apparatus 1 according to the present embodiment can maintain the duty ratio of each switching clock even when the switching frequency is changed in a cycle based on the PRI. FIG. 5 is a diagram for illustrating the duty ratio of each switching clock of the case where the control circuitry 23 according to the present embodiment changes the switching frequency. The upper waveform shown in FIG. 5 shows the transmission waveform of ultrasound pulses. The lower waveform shown in FIG. 5 shows the transmission waveform of switching clocks. Specifically, the lower waveform shows the transmission waveform of switching clocks S1, S2, and S3. t=t5, and t=t6 indicate times when the switching frequency is changed. In FIG. 5, the control circuitry 23 changes the switching frequency based on the transmission timing T of ultrasound pulses. According to FIG. 5, the duty ratios of switching clocks S1, S2, and S3 are t11/t12, t21/t22, and t31/t32, respectively.

In general, when a switching clock is reset near the transmission timing T of an ultrasound pulse, the duty ratio of the switching clock may not be maintained. If the duty ratio of the switching clock is not maintained, the output voltage of the switching power supply 25 becomes unstable. The control circuitry 23 according to the present embodiment gradually changes the switching frequency by, for example, 1% of the switching frequency before the change, and can control the values of t11/t12, t21/t22, and t31/t32 to be equal to one another. Therefore, the switching power supply 25 can supply a stable output voltage to, for example, the ultrasound transmission circuitry 11 and the ultrasound reception circuitry 12.

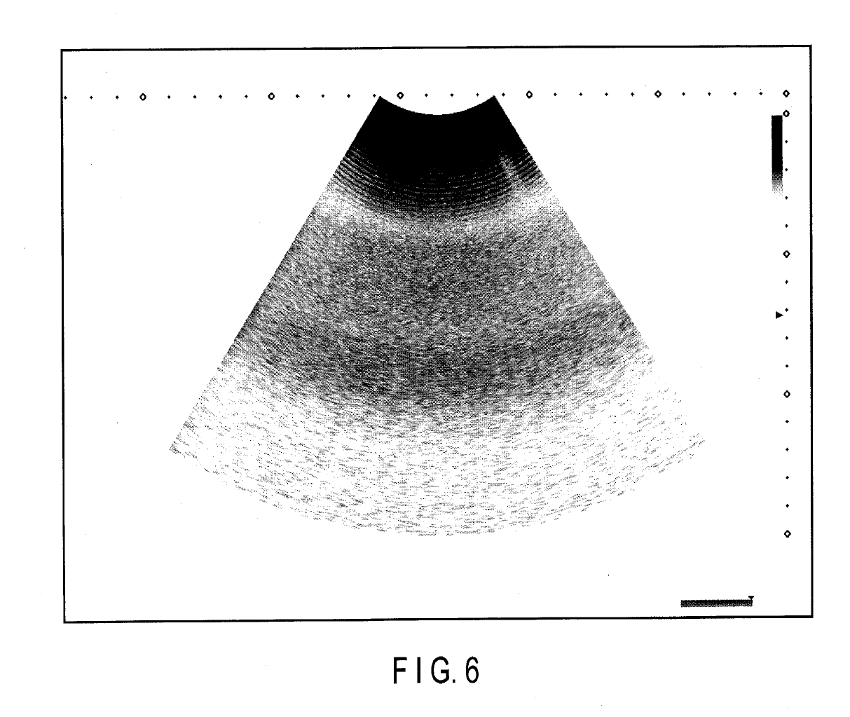

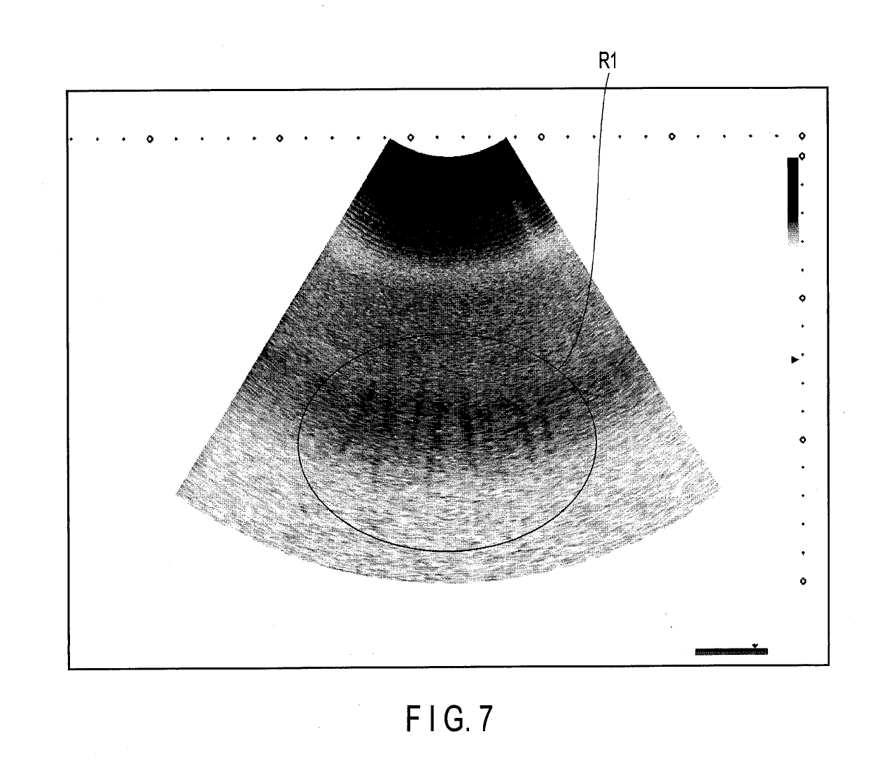

Next, the ability to inhibit the switching noise shown on the ultrasound image generated by the method for controlling the switching frequency by the control circuitry 23 according to the present embodiment will be explained below with reference to drawings. The following description will be provided while assuming that a combination focus is performed on four transmission focuses in a B-mode scan, whereby a B-mode image is generated. FIG. 6 is a diagram showing an example of the B-mode image displayed by the ultrasound diagnostic apparatus 1 according to the present embodiment on the display 50. FIG. 7 is a diagram showing an example of the B-mode image of the case where the switching frequency is not controlled. In general, a switching noise appears parallel to the scan line (raster) direction. In FIG. 6, no prominent switching noise is shown on the B-mode image. In FIG. 7, a prominent switching noise is shown parallel to the scan line (raster) direction in region R1. In this way, the ultrasound diagnostic apparatus 1 according to the present embodiment can suppress the switching noise shown on the B-mode image.

According to the above-described embodiment, the control circuitry 23 controls the ultrasound transmission circuitry 11 and the ultrasound reception circuitry 12, transmits ultrasound to the subject P in a cycle based on the PRI, and receives reflected wave signals from the subject P. The switching power supply 25 generates a voltage by switching in accordance with the switching frequency of switching clocks supplied by the control circuitry 23, and supplies the generated voltage to the ultrasound transmission circuitry 11 and the ultrasound reception circuitry 12. The control circuitry 23 changes the switching frequency by predetermined change widths in the cycle based on the PRI.

Accordingly, even when a plurality of received signals generated by, for example, a B-mode scan, or an M-mode scan are summed, switching noises can be inhibited from being summed in the same phase because the phase difference between the PRF and switching frequencies are scattered.

Therefore, when a B-mode scan or an M-mode scan is performed, an increase in the switching noise shown on the ultrasound image can be inhibited.

### Second Embodiment

Described in the first embodiment is the case where the ultrasound transmission circuitry 11, the ultrasound reception circuitry 12, the signal processing circuitry 13, the control circuitry 23, and the switching power supply 25 are included in the apparatus main body 10. However, the configuration is not limited to this. Described in the second embodiment is the case where the ultrasound transmission circuitry 11, the ultrasound reception circuitry 12, the signal processing circuitry 13, the control circuitry 23, and the switching power supply 25 are included in an ultrasound probe 70a.

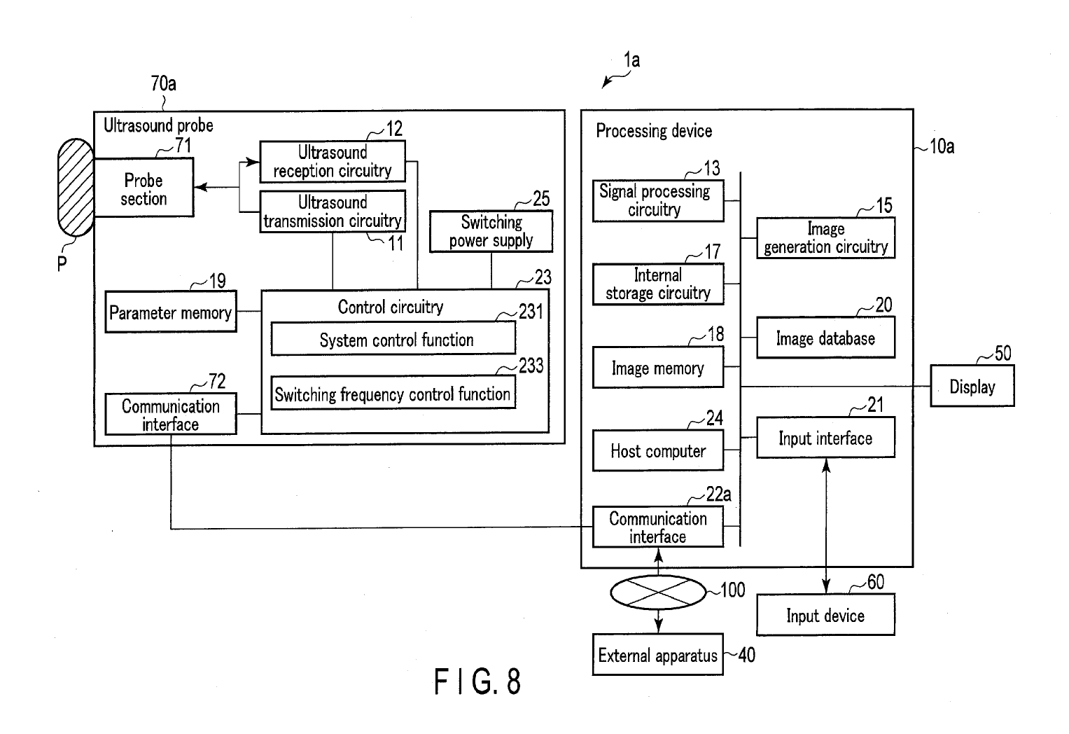

FIG. 8 is a diagram showing an example of the functional configuration of an ultrasound diagnostic apparatus 1a according to the second embodiment. As shown in FIG. 8, the ultrasound diagnostic apparatus 1a includes a processing device 10a, an ultrasound probe 70a, a display 50, and an input device 60. The processing device 10a is connected to an external apparatus 40 via a network 100. The processing device 10a is also connected to the display 50 and the input device 60. The ultrasound probe 70a is detachably attached to the processing device 10a.

The ultrasound probe 70a includes a probe section 71, ultrasound transmission circuitry 11, ultrasound reception circuitry 12, a communication interface 72, a parameter memory 19, control circuitry 23, and a switching power supply 25. The ultrasound probe 70a may include, as an input interface, a button or the like that is pressed, for example, when performing offset processing or when freezing an ultrasound image.

The probe section 71 includes, for example, a plurality of piezoelectric vibrators, a matching layer provided in each piezoelectric vibrator, and a backing material for preventing backward propagation of ultrasound from the piezoelectric vibrators. The probe section 71 generates ultrasound by the piezoelectric vibrators, based on a drive signal supplied from ultrasound transmission circuitry 11. When ultrasound is transmitted from the probe section 71 to the subject P, the transmitted ultrasound is reflected by one after another of discontinuous surfaces having acoustic impedances in body tissue of the subject P. The probe section 71 receives the reflected waves by the piezoelectric vibrators. The probe section 71 converts the received reflected waves into a reflected wave signal.

The communication interface 72 is connected to the processing device 10a by wire or radio, and performs data communication with the processing device 10a Specifically, the communication interface 72, for example, receives various instructions from the host computer 24 of the processing device 10a, and outputs the received instructions to the control circuitry 23. The communication interface 72 also outputs the received signal generated by the ultrasound reception circuitry 12 to the processing device 10a. The wired connection is realized by, for example, a universal serial bus (USB), but is not limited to this.

The processing device 10a shown in FIG. 8 is a device that generates an ultrasound image based on the received signal output from the ultrasound probe 70a The processing device 10a includes signal processing circuitry 13, image generation circuitry 15, internal storage circuitry 17, an image memory 18, an image database 20, an input interface 21, a communication interface 22a, and a host computer 24.

The communication interface 22a is connected to the ultrasound probe 70a by wire or radio, and performs data communication with the ultrasound probe 70a. Specifically, the communication interface 22a outputs various instructions from the host computer 24 to the ultrasound probe 70a, for example. The communication interface 22a outputs the received signal generated at the ultrasound probe 70a to the host computer 24. The communication interface 22a is connected to the external apparatus 40 via, for example, the network 100, and performs data communication with the external apparatus 40.

The configurations of the ultrasound probe 70a and the processing device 10a are not limited to the above. For example, the ultrasound probe 70a may not necessarily include the parameter memory 19. The ultrasound probe 70a may include signal processing circuitry 13. The ultrasound probe 70a may include a memory storing, for example, a control program for executing ultrasound transmission and reception, and a control program for realizing the switching frequency control function 233.

All the structures included in the apparatus main body 10a of the present embodiment may be included in the ultrasound probe 70a. In this case, the ultrasound probe 70a may be connected, by a USB or radio, to a display 50 (such as a display, a tablet terminal, or a smart phone) for displaying an ultrasound image thereon.

The processing device 10a may include the display 50 and the input device 60. In this case, the processing device 10a is realized by a terminal apparatus, such as a tablet terminal or a smart phone.

### Other Embodiments

In addition, the functions of the embodiments may also be realized by installing programs that execute respective processes in a computer, such as a work station, and loading them in the memory. The programs that can cause the computer to perform the methods may be distributed by being stored in storage media such as a magnetic disk (such as a hard disk), an optical disk (such as a CD-ROM, or a DVD), and a semiconductor memory.

According to at least one embodiment described above, when a B-mode scan or an M-mode scan is performed, an increase in the switching noise shown on the ultrasound image can be inhibited.

The term “processor” used in the above description means, for example, a central processing unit (CPU), a graphics processing unit (GPU), or circuitry such as an application specific integrated circuit (ASIC), a programmable logic device (e.g., a simple programmable logic device (SPLD), a complex programmable logic device (CPLD), or a field programmable gate array (FPGA). The processor realizes a function by reading and executing a program stored in the memory circuitry. Each processor of the above-described embodiments is not necessarily configured as a single circuit, and a plurality of independent circuits may be configured in combination as one processor to realize the function. In addition, a plurality of structural elements in FIG. 1 may be integrated in one processor to realize the function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasound diagnostic apparatus, comprising:
a transmission/reception circuitry that transmits ultrasound to a subject in a predetermined repetition cycle and receives an echo signal from the subject;
a switching power supply that generates a voltage by switching in accordance with a switching frequency, and supplies the transmission/reception circuitry with the voltage; and
control circuitry that changes the switching frequency by a predetermined change width in the predetermined repetition cycle.

2. The ultrasound diagnostic apparatus according to claim 1, wherein
the control circuitry gradually changes the switching frequency in the predetermined repetition cycle.

3. The ultrasound diagnostic apparatus according to claim 1, wherein
the control circuitry changes the switching frequency by a width that inhibits the supplied output voltage from being unstable in the predetermined repetition cycle.

4. The ultrasound diagnostic apparatus according to claim 1, wherein
the control circuitry changes the switching frequency by 1% in the predetermined repetition cycle.

5. An ultrasound probe, comprising:
a transmission/reception circuitry that transmits ultrasound to a subject in a predetermined repetition cycle and receives an echo signal from the subject;
a switching power supply that generates a voltage by switching in accordance with a switching frequency, and supplies the transmission/reception circuitry with the voltage; and
control circuitry that changes the switching frequency by a predetermined change width in the predetermined repetition cycle.

6. The ultrasound probe according to claim 5, wherein
the control circuitry gradually changes the switching frequency in the predetermined repetition cycle.

7. The ultrasound probe according to claim 5, wherein
the control circuitry changes the switching frequency by a width that inhibits the supplied output voltage from being unstable in the predetermined repetition cycle.

8. The ultrasound probe according to claim 5, wherein
the control circuitry changes the switching frequency by 1% in the predetermined repetition cycle.
